(19) United States
(12) Patent Application Publication
(10) Pub. No.: US 2006/0182787 A1
(43) Pub. Date: Aug. 17, 2006

(54) Skin or wound pad containing encapsulated substances which promote the healing of wounds and/or are used for skin care

(75) Inventors: Jan Jaenichen, Hamburg (DE); Guenther Sachau, Quickborn (DE); Carsten Hartkopf, Hamburg (DE); Thorsten Berg, Malang (ID)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 11/326,479

(22) Filed: Jan. 6, 2006

Related U.S. Application Data
(63) Continuation of application No. PCT/EP04/05052, filed on May 12, 2004.

(30) Foreign Application Priority Data
Jul. 8, 2003 (DE) 103 30 971.3

(51) Int. Cl. A61F 5/00; A61L 15/00

(52) U.S. Cl. 424/445; 602/1

(57) ABSTRACT

A skin or wound contact material which comprises a polycondensate matrix and a water-absorbing polymer incorporated therein. The water-absorbing polymer is doped with a wound healing promoter substance and/or a skin care substance.

### CROSS-REFERENCE TO RELATED APPLICATIONS

The present application is a continuation of International Application No. PCT/EP2004/005052, filed May 12, 2004, the entire disclosure whereof is expressly incorporated by reference herein, which claims priority of German Patent Application No. 103 30 971.3, filed Jul. 8, 2003.

### BACKGROUND OF THE INVENTION

1. Field of the Invention

The present invention relates to a skin or wound contact material or pad with wound healing promoter substances and/or skin care substances which are present in encapsulated form in the material and which controllably deliver the substance only with contact with the wound secretion or moisture.

2. Discussion of Background Information

Correct pharmaceutical formulation of physiological actives is one of the principal tasks of the pharmaceutics in the drug industry. Thus the actual active substance in the pharmaceutical formulation often accounts for only a small part of the overall formula, alongside numerous physiologically inactive excipients. However, it is these very excipients via which release location and release kinetics of an active substance in the body can be modified so as to produce optimum development of the desired action. For instance, acid-resistant capsules can be used to ensure that active substances pass through the acidic stomach before being released in a targeted manner in the alkaline environment of the intestine; delayed-release formulations utilize the diffusion-controlled release of the active substance from the pharmaceutical in order to make the active substance available at pharmacologically active concentrations over a prolonged period (in this regard see also K. H. Bauer, K-H. Frömming, C. Führer in “Pharmazeutische Technologie”, 5th edition 1997).

Skin injuries and wounds pose an interesting challenge to the development of suitable formulations of wound healing promoter substances, since not only the high level of metabolic activity in the wound but also the steady secretion of wound fluid ensure a continuously changing wound milieu. Formulations with wound healing promoter substances and disinfectant substances are known in particular on the basis of liquid formulas or ointments and must be applied a number of times a day for optimum development of action.

Treating wounds with wound plasters and wound dressings has the aim, primarily, of preventing mechanical penetration of foreign bodies and microorganisms and of creating a wound milieu in which optimum conditions prevail for the healing process of the skin.

Modern wound care products such as hydrocolloids (in this regard see, for example, “Hydrokolloide” by R. Lipmann in Medical Device & Diagnostic Industry, June 1999), which were developed for colostomy applications and occupational wound care applications, are finding application increasingly.

Wound care products based on hydrocolloids have advantages over conventional plasters. They generate a moist wound healing environment, which does not let the wound dry out and produces an optimum environment for rapid wound healing. Further advantages are the inconspicuousness in application, secure adhesion, absorption capacity for exudate, effective cushioning, and painless removability.

Contoured wound contact materials with an adhesive layer composed of swellable hydrocolloids and water-insoluble viscous constituents, examples being polyisobutylene, rubber, silicon or polyurethane elastomers, are subject matter of WO 92/05755.

Water-free hydrogels are referred to as xerogels and are macromolecular, natural or synthetic substances which by virtue of a high level of hydrophilic groups are capable of binding water absorptively. The water absorption capacity of many xerogels is a multiple of the intrinsic weight of the water-free substance. Hydrogels or xerogels are employed in diverse form in wound care, since they protect wounds against drying out, draw up wound secretion, and serve as a matrix for active substances of all kinds and also as a basis for population with autologous or heterologous skin cells.

One form in which gels can be used is that of foams. Foams for treating skin wounds or surgical wounds are known per se to the skilled worker. In this context use is made predominantly of polyurethane foams or collagen foams.

Self-adhesive gel foams as well are known to the skilled worker. Although these foams generally attach very well to the skin, in the majority of cases they have the drawback that their water absorption capacity and water retention capacity are severely restricted.

Furthermore, hydrophilic foams of polyurethane gels are known. WO 88/01878 A1 describes self-adhesive polyurethane foams or polyurethane foam gels which can include, among other monomer units, copolymerized methacrylates. These foam gels are produced by adding water.

Polyurethane gels based on a polyurethane matrix and relatively high molecular mass polyols are also described in EP 0 057 839 B1. Self-attaching sheet-like structures comprising polyurethane gels are known from EP 0 147 588 B1. The polyurethane gels disclosed in these two last-mentioned texts are unfoamed. The self-adhesive gels have isocyanate indexes of 15 to 70 (EP 0 147 588 A2).

EP 0 196 364 A2 describes hydrophilic polyurethane foams which may be filled with water-absorbing polymers based on a copolymer of acrylic acid and potassium acetate and are intended for medical use. The polyurethane is prepared on the basis of MDI, methylenediphenyl diisocyanate. The polyether used has a minimum functionality of two hydroxyl groups, preferably two to three hydroxyl groups in each case. The NCO/OH ratio is stoichiometric. The polyurethane is accordingly not gel-like. Foaming can be carried out using pressurized air or using other gases which do not react with the isocyanate, or by means of low-boiling solvents. Water-absorbing polymer and polyether polyol are mixed in a ratio of about 3:1, serving for water absorption. The foam has adhesive properties on wounds, which have to be eliminated completely by means of an aluminized web in order that the foam may be used for wound treatment. The water-absorbing polymers disclosed in EP 0 196 364 A2 are not doped with actives.

Foam wound contact materials composed of a polyurethane gel foam comprising a polyaddition product of a polyether polyol (Levagel®, Bayer AG) with an aromatic or aliphatic diisocyanate (Desmoder®, Bayer AG), into which a polyacrylate superabsorbant powder (Favor®, Stockhausen) has been incorporated, are described inter alia in DE 42 33 289 A1, in DE 196 18 825 A1, and WO 97/43328. Depending on the ratio of OH equivalents in the polyol to reactive isocyanate groups, the polyurethane gel may be formulated for weak or strong self-attachment to skin.

It is known that the incorporation of water-absorbing polymers into the reactive precursors of a polyurethane reaction allows the preparation of polyurethane-based wound contact materials which have excellent skin compatibility and also a high capacity to absorb fluid. The moist wound milieu promoted by wound contact materials of this kind contributes to a considerable acceleration in wound healing. In none of the prior art publications, however, are absorbents doped with wound healing promoter actives or with skin care substances disclosed in polycondensate matrices, especially polyurethane matrices.

Furthermore, active substance patch systems in the form of transdermal therapeutic systems (TTS) for delivering active substances through the skin have been known for a long time. The topical application of drugs by way of active substance patch systems offers two main advantages: First, this form of administration produces first-order release kinetics of the active substance, thereby enabling a constant level of active substance to be maintained in the body over a fairly long time period. Secondly, the path of uptake through the skin avoids the gastrointestinal tract and also the first liver passage. As a result, selected drugs may be effectively administered in a low dose. This is particularly advantageous when the drug is desired to act locally while avoiding a systemic effect. This is the case, for example, with the treatment of rheumatic joint complaints or muscular inflammation.

One embodiment of such transdermal systems which has been well described in the technical literature is that of matrix systems or monolithic systems, in which the drug is incorporated directly into the pressure-sensitive adhesive. In the ready-to-apply product, a pressure-sensitively adhesive matrix comprising active substance of this kind is equipped on one side with a backing impermeable to the active substance, while on the opposite side there is a backing film equipped with a release layer, which is removed prior to application to the skin (kleben&dichten, No. 42, 1998, pp. 26 to 30). For instance, the use of polyacrylates and/or polyurethanes is mentioned principally as a basis for the pressure-sensitively adhesive polymer matrix (Lamba, Woodhouse, Cooper, “Polyurethanes in Biomedical Applications”, CRC Press, 1998, p. 240) and WO 01/68060.

A problem associated with the production of transdermal therapeutic systems is the introduction of polar active substances into the usually nonpolar polymer matrices. As a result, preferred active substances may occasionally be incorporated with difficulty or in limited concentration into the polymer matrix. Furthermore, there is a risk, owing to the difference in polarity and the insolubility of the active substances in the polymer matrix, that the active substances will crystallize out of the polymer system over time. Long-term stability is hence not always guaranteed.

With regard to the incorporation of different wound healing promoter substances or skin care substances into matrices, especially polyurethane matrices, the problem exists, furthermore, that many of these substances also undergo reaction with polar functional groups, such as dexpanthenol, for example, in the crosslinking reaction of the polyurethane. As a result, on the one hand, there is disruptive crosslinking of the matrix, and on the other hand the active substance is also incorporated covalently into the matrix, and can no longer be released from it. Consequently it is impossible to incorporate these active substances into the matrix prior to the crosslinking reaction. It is therefore necessary to introduce these substances into the polyurethane matrix in costly and inconvenient downstream operations. This is disruptive not only for the production step.

Known from the prior art, furthermore, are a series of documents which disclose water-absorbing polymers doped with active substance. For instance, US 2003/0004479 describes a water-absorbing composition composed of a water-absorbing polymer and a plant powder active, the water-absorbing polymer being post-crosslinked in the surface region.

It would be desirable to be able to provide a skin or wound contact material capable of absorbing moisture, especially water, and of delivering a wound healing promoter and/or skin care active. It would hence also be desirable to be able to provide a contact material capable of caring for human skin, increasing its resistance and/or healing a wound.

It would in particular be desirable to provide a wound dressing capable of absorbing wound exudate that is adequately able to draw up moisture from the skin and, where appropriate, to transport it outward through the plaster, that generates a moist wound healing environment, that is skin-compatible, that is painlessly redetachable, and that comprises wound healing promoter and/or skin care adjuvants which can be delivered in a controlled way over a prolonged time period.

It would further be desirable to provide a contact material which through the addition of wound healing promoter substances and/or skin care substances does not exert any influence on the possibly self-adhesive properties of the contact material, and which is simple and inexpensive to produce.

### SUMMARY OF THE INVENTION

The present invention provides skin or wound contact material which comprises a polycondensate matrix and a water-absorbing polymer incorporated therein. The water-absorbing polymer is doped with a wound healing promoter substance and/or a skin care substance.

In one aspect of the material, at least a part of the water-absorbing polymer may be covalently bonded to the polycondensate matrix.

In another aspect, the polycondensate matrix may be air and water vapor permeable and/or self-adhesive and/or transparent.

In yet another aspect, the polycondensate matrix may comprise a polyurethane matrix. By way of non-limiting example, the polyurethane matrix may be formed from
(a) one or more polyether polyols having from 2 to 6 hydroxyl groups, OH numbers of from 20 to 112, and an ethylene oxide content of at least 10% by weight,
(b) one or more antioxidants,
(c) a catalyst comprising one or more bismuth(III) carboxylates which are based on carboxylic acids having from 2 to 18 carbon atoms and are soluble in the polyols (a), and
(d) hexamethylene diisocyanate.

In one aspect of the above polyurethane matrix, the product of the functionalities of components (a) and (d) may be at least 5.2 and the ratio of free NCO groups of component (d) to free OH-groups of component (a) may be from 0.30 to 0.70 and/or component (c) may be present in an amount of from 0.005% to 0.25% by weight and/or component (b) may be present in an amount of from 0.1% to 1.0% by weight, each based on component (a).

In another aspect of the material of the present invention, the water-absorbing polymer may be present in particulate form.

In yet another aspect of the material, the water-absorbing polymer may comprise at least 50% by weight, e.g., at least 70% by weight, or at least 90% by weight, of one or more carboxylate group containing monomers.

In a still further aspect of the above material, the water-absorbing polymer may comprise at least 50% by weight of acrylic acid, and at least 20 mol %, e.g., at least 50 mol %, and preferably from 65 to 85 mol % of the acrylic acid may be neutralized.

In another aspect, the water-absorbing polymer may comprise crosslinked sodium polyacrylate.

In another aspect of the material of the material of the present invention, the water-absorbing polymer may exhibit one or more of the following properties:
A1) a particle size distribution wherein at least 80% by weight of particles have a size of from 10 μm to 900 μm, determined according to ERT 420.1-99;
A2) a centrifuge retention capacity (CRC) of at least 10 g/g, e.g., at least 20 g/g, determined according to ERT 441.1-99;
A3) an absorption against pressure (AAP) at 0.7 psi of at least 4 g/g, determined according to ERT 442-1-99;
A4) a water-soluble polymer content after a 16 hour extraction of less than 25% by weight, based on the total weight of the water-absorbing polymer, determined according ERT 470.1-99;
A5) a residual moisture content of not more than 15% by weight, based on the total weight of the water-absorbing polymer, determined according ERT 430.1-99.

In yet another aspect of the material of the present invention, the water-absorbing polymer may exhibit at a particle size distribution of from 10 μm to 500 μm and/or a residual moisture content of less than 10% by weight, e.g., less than 3% by weight.

In a still further aspect of the material, the wound healing promoter substance and/or skin care substance may be present in an amount of from 0.001% to 30% by weight, e.g., from 5% to 15% by weight, based on the total weight of the water-absorbing polymer plus the wound healing promoter substance and/or skin care substance.

In another aspect, the wound healing promoter substance and/or skin care substance may be present in an amount of from 0.1% to 10.0% by weight, e.g., from 0.2% to 5% by weight, based on the weight of the matrix.

In another aspect of the material, the wound healing promoter substance and/or skin care substance may be distributed, preferably homogeneously, over the entire water-absorbing polymer.

In yet another aspect of the material, the water-absorbing polymer may be present in an amount of from 70% to 99.99% by weight, based on the total weight of the water-absorbing polymer and the wound healing promoter substance and/or skin care substance, the water-absorbing polymer may comprise at least 90% by weight of a crosslinked polyacrylic acid, based on the water-absorbing polymer, and the crosslinked polyacrylic acid may comprise at least 90% by weight, based on the crosslinked polyacrylic acid, of acrylic acid which may comprise at least 30 mol % of partially neutralized acrylic acid.

In a still further aspect of the material, the wound healing promoter substance and/or skin care substance may exhibit an availability of at least 10% by weight, determined according to the extraction test described hereinafter.

In yet another aspect of the material of the present invention, the wound healing promoter substance and/or skin care substance may comprise one or more of dexpanthenol, marigold, witch hazel, camomile, a vitamin, an antioxidant, a light stabilizer, an insect repellent, an essential oil, an antimicrobial agent, a moisturizer, a perfume and coenzyme Q10, preferably at least dexpanthenol and/or coenzyme Q10.

The present invention also provides a skin or wound contact material which comprises a self-adhesive, air and water vapor permeable polyurethane matrix and a water-absorbing polymer incorporated therein. The water-absorbing polymer comprises at least 50% by weight of one or more carboxylate group containing monomers and is doped with a wound healing promoter substance and/or a skin care substance.

In one aspect of the material, at least a part of the water-absorbing polymer may be covalently bonded to the polyurethane matrix and/or the water-absorbing polymer may be present in particulate form.

In another aspect, the water-absorbing polymer may comprise at least 50% by weight of acrylic acid and from 65 to 85 mol % of the acrylic acid may be neutralized.

In yet another aspect, the water-absorbing polymer may exhibit a particle size distribution of from 10 μm to 500 μm and/or a residual moisture content of less than 3% by weight.

In a still further aspect, the wound healing promoter substance and/or skin care substance may comprise at least one of dexpanthenol, marigold, witch hazel, camomile, a vitamin, an antioxidant, a light stabilizer, an insect repellent, an essential oil, an antimicrobial agent, a moisturizer and coenzyme Q10.

In yet another aspect of the materials of the present invention, the materials may further comprise a backing sheet. For example, the matrix may be applied in foamed or unfoamed form, partially or over the whole area, to the backing sheet. By way of non-limiting example, the backing sheet may comprise at least one of a polyurethane, a polyethylene, a polypropylene, a polyamide, a polyester and a polyether-ester.

In another aspect, the materials may further comprise a liner sheet and/or a liner paper and/or a release paper.

In another aspect, the materials of the present invention may be comprised in a wound dressing, a bandage or a plaster, or they may be comprised in a dry or moist cosmetic wipe or a pad.

The present invention also provides a process for producing a skin or wound contact material which comprises a polyurethane matrix and a water-absorbing polymer which has a wound healing promoter substance and/or a skin care substance incorporated therein. The process comprises reacting a mixture comprising a polyether polyol and an aliphatic isocyanate prepolymer and adding a water-absorbing polymer doped with the wound healing promoter substance and/or skin care substance to form the polyurethane matrix having the water-absorbing polymer incorporated therein.

In one aspect, the process may further comprise the subsequent coating of the polyurethane matrix having the water-absorbing polymer incorporated therein two-dimensionally onto a backing sheet.

It was surprising, and unforeseeable for the skilled worker, that a skin or wound contact material comprising
a. a polycondensate matrix, preferably a polyurethane matrix, based on at least one polycondensable monomer having at least one polycondensable group, and
b. a particulate, water-absorbing polymer comprising at least one wound healing promoter substance and/or at least one skin care substance which has at least one functional group that is able to react with the polycondensable group and forms a covalent bond with the polycondensable group, or

c. a water-absorbing polymer comprising at least one wound healing promoter substance and/or at least one skin care substance,

the particulate, water-absorbing polymer being at least partly surrounded by the polycondensate matrix,

at least the particulate, water-absorbing polymer comprising the wound healing and/or skin care substance, and

the skin or wound contact material exhibiting a wound-healing substance or active substance availability of at least 10% by weight by the extraction test indicated herein achieves the above objects.

In particular, a skin or wound contact material comprising an air and water vapor permeable, preferably self-adhesive polyurethane matrix comprising a water-absorbing polymer into which at least one wound healing promoter substance and/or at least one skin care substance, also referred to below as active substance, is incorporated achieves the stated objects and remedies the disadvantages of the prior art.

By “water-absorbing” is meant in accordance with the invention not only the capacity of a substance to take up water into itself, with formation of a hydrogel, but also any absorption of aqueous fluids, especially aqueous body fluids such as urine, blood, blood constituents such as pus, lymph fluids or blood serum.

Polycondensates used in accordance with the invention are preferably polyurethanes. In general, polyurethanes are prepared from the known starting compounds of polyurethane chemistry by known processes, which are set forth in DE-A 3103499, DE-A 3103500, EP 0 147 588 A1, EP 0 665 856 B1 or DE 196 18 825 A1.

Polyurethane is used as a basis for the active substance matrix. The polyurethane (c) is prepared by polymerizing an alcohol (a) with an isocyanate (b).

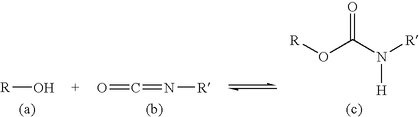

A decisive advantage of the polyurethane polymer or gel matrices are their self-adhesive properties, which make it unnecessary additionally to apply an adhesive layer to the matrix in order to attach the wound dressing in the region of the skin. At its most simple the active substance polyurethane matrix is located between a cover layer firmly anchored to it, also dubbed backing layer, and a removable release layer.

The purpose of the removable release layer is to secure the adhesive layer and to improve stability in transit and on storage, and it is removed prior to application to the skin.

The polyurethane matrix may be applied to a backing layer or backing sheet of the kind known from the prior art. The backing sheet is composed of an air and water vapor permeable but water impermeable polymer layer having a thickness of approximately 10 to 100 μm. The backing sheet, flexible under certain circumstances, is composed preferably of polymers of polyurethane, PE, PP, polyamide, polyester or polyether-ester.

Suitable polyurethane matrices are subject matter of DE 196 18 825, which discloses hydrophilic, self-adhesive polyurethane gels composed of
a) polyether polyols having from 2 to 6 hydroxyl groups, OH numbers of from 20 to 112, and an ethylene oxide (EO) content of >10% by weight,
b) antioxidants,
c) bismuth (III) carboxylates soluble in the polyols (a) and based on carboxylic acids having from 2 to 18 carbon atoms, as catalysts, and
d) hexamethylene diisocyanate,
   with a product of the functionalities of the polyurethane-forming components a) and d) of at least 5.2, the amount of catalyst c) being from 0.005% to 0.25% by weight, based on the polyol a), the amount of antioxidants b) being in the range from 0.1% to 1.0% by weight, based on polyol a), and the selected ratio of free NCO groups of component d) to the free OH— groups of component a) (isocyanate index) being in the range from 0.30 to 0.70.

It is preferred to use polyether polyols having 3 to 4, very preferably 4 hydroxyl groups, with an OH number in the range from 20 to 112, preferably from 30 to 56. The ethylene oxide content of the polyether polyols employed in accordance with the invention is preferably ≧20% by weight.

The polyether polyols as such are known per se and are prepared for example by polymerizing epoxides, such as ethylene oxide, propylene oxide, butylene oxide or tetrahydrofuran, with themselves or by addition reaction of these epoxides, preferably of ethylene oxide and propylene oxide, where appropriate in a mixture with one another or separately in succession, with starter components having at least two reactive hydrogen atoms, such as water, ethylene glycol, propylene glycol, diethylene glycol, dipropylene glycol, glycerol, trimethylolpropane, pentaerythritol, sorbitol or sucrose. Representatives of the stated high molecular mass polyhydroxyl compounds for use are listed for example in High Polymers, vol. XVI, “Polyurethanes, Chemistry and Technology” (Saunders-Frisch, Interscience Publishers, New York, vol. 1, 1962, pp. 32-42).

As an isocyanate component use may be made of monomeric or trimerized hexamethylene diisocyanate or of hexamethylene diisocyanate modified by biuret, uretdione or allophanate groups or by prepolymerizing with polyether polyols or with mixtures of polyether polyols based on the known starter components having 2 to >2 reactive H atoms and epoxides, such as ethylene oxide or propylene oxide with an OH number of ≦850, preferably from 100 to 600. Preference is given to using modified hexamethylene diisocyanate, especially hexamethylene diisocyanate modified by prepolymerization with polyether diols of OH number from 200 to 600. Very particular preference is given to modifications of hexamethylene diisocyanate with polyether diols with an OH number of 200-600 whose residual monomeric hexamethylene diisocyanate content is below 0.5% by weight.

Catalysts suitable for the polyurethane gels of the invention are bismuth (III) carboxylates soluble in the anhydrous polyether polyols a) and based on linear, branched, saturated or unsaturated carboxylic acids having from 2 to 18, preferably from 6 to 18, C atoms. Preference is given to Bi(III) salts of branched saturated carboxylic acids having tertiary carboxyl groups, such as of 2,2-dimethyloctanic acid (for example, Versatic acids, Shell). Highly suitable preparations are preparations of these Bi(III) salts in excess fractions of these carboxylic acids. A system which has been found outstandingly appropriate is a solution of 1 mol of the Bi(III) salt of Versatic 10 acid (2,2-dimethyloctanic acid) in an excess of 3 mol of this acid having a Bi content of about 17%.

The catalysts are used preferably in amounts of from 0.03% to 0.3% by weight, based on the polyol a).

Suitable antioxidants for the polyurethane gels of the invention are, in particular, sterically hindered phenolic stabilizers, such as BHT (2,6-di-tert-butyl-4-methylphenol), Vulkanox BKF (2,2 min methylene-bis-(6-tert-butyl-4-methylphenol) (Bayer AG), Irganox 1010 (pentaerythrityl tetrakis[3-(3,5-di-tert-butyl-4-hydroxyphenol)propionate]), Irganox 1076 (octadecyl 3-(3,5-di-tert-butyl-4-hydroxyphenol)propionate) (Ciba-Geigy) or tocopherol (vitamin E). Preference is given to using those of the α-tocopherol type. The antioxidants are used preferably in amounts of from 0.15 to 0.5% by weight, based on the polyol a).

The isocyanate index (ratio of the free NCO groups used in the reaction to the free OH groups) of the polyurethane gel compositions of the invention is in the range from 0.30 to 0.70, preferably in the range from 0.45 to 0.60, depending on the functionality of the isocyanate components and polyol components employed. The isocyanate index required for gel formation can be estimated very simply by the following formula:

$${f}_{( polyol )} \cdot ( {f}_{( isocyanate )} - 1 ) \cdot Index \approx 2$$

$$Index \approx \frac{2}{{f}_{( polyol )} * ( {f}_{( isocyanate )} - 1 )}$$

f: functionality of isocyanate or polyol component

Depending on the target tackiness or elasticity of the gel, the isocyanate index to be actually used may differ by up to ±20% from the calculated value.

The polyurethane gel compositions of the invention are produced by customary processes such as are described for example in Becker/Braun, Kunststoff-Handbuch, vol. 7, Polyurethane, p. 121 if, Carl-Hauser, 1983.

With further preference polyurethane gels are employed as are disclosed in EP 0 665 856 B1. The hydrophilic polyurethanes are obtainable accordingly from

1. a polyurethane gel which comprises

(A) 25-62%, preferably 30-60%, more preferably 40-57%, by weight based on the sum of (A) and (B), of a covalently crosslinked polyurethane as a high molecular weight matrix and
(B) 75-38%, preferably 70-40%, more preferably 60-43%, by weight based on the sum of (A) and (B), of one or more polyhydroxyl group compounds which are firmly held in the matrix by secondary valence forces and have an average molecular weight of between 1000 and 12 000, preferably between 1500 and 8000, more preferably between 2000 and 6000, and an average OH number of between 20 and 112, preferably between 25 and 84, more preferably between 28 and 56, as a liquid dispersant, the dispersant being substantially free of hydroxyl compounds having a molecular weight of below 800, preferably below 1000, more preferably below 1500, and also, if desired
(C) 0 to 100% by weight, based on the sum of (A) and (B), of fillers and/or additives,
   and which is obtainable by reacting a mixture of
a) one or more polyisocyanates
b) one or more polyhydroxyl compounds having an average molecular weight of between 1000 and 12 000 and an average OH number of between 20 and 112,
c) if desired, catalysts or accelerators for the reaction between isocyanate groups and hydroxyl groups, and also, if desired,
d) fillers and additives known per se from polyurethane chemistry,
this mixture being substantially free of hydroxyl compounds having a molecular weight below 800, the average functionality of the polyisocyanates (Fᵢ) being between 2 and 4, the average functionality of the polyhydroxyl compound (Fₚ) being between 3 and 6, and the isocyanate index (K) conforming to the formula

$$K = \frac{300 \pm X}{( {F}_{i} \cdot {F}_{p} ) - 1} + 7$$

in which X is ≦120, preferably X is ≦100, more preferably X is ≦90, and the index K is at values between 15 and 70, the stated molecular weight and OH number averages being number averages,
2. a water-absorbing material and/or
3. a nonaqueous foaming agent.

With regard to the preparation of preferably self-adhesive polyurethane it should be borne in mind that the above-defined conditions are complied with when the gel-forming components are selected, since otherwise tack-free elastic gels rather than self-adhesive gels are obtained.

Preferred hydroxyl compounds are polyether polyols as specified at length in the abovementioned laid-open specifications.

Suitable polyisocyanate components include not only (cyclo)aliphatic but also aromatic isocyanates. Preferred (cyclo)aliphatic polyisocyanates are 1,6-hexamethylene diisocyanate and also its biurets and trimers and hydrogenated diphenylmethane diisocyanate (“MDI”) grades. Preferred aromatic polyisocyanates are those which are obtained by distillation, such as MDI mixtures of 4,4′ and 2,4′-isomers or 4,4′-MDI, and also toluene diisocyanate (“TDI”) grades.

The diisocyanates may be selected in particular, for example, from the group of the unmodified aromatic or aliphatic diisocyanates or else from modified products formed by prepolymerization with amines or polyols, including polyether polyols.

As advantages of the polyurethanes of the invention in comparison to other polycondensates and polymers, used in particular for the production of dressing materials, the following points may be cited:
Polyurethane can be provided flexibly as a self-adhesive or nonadhesive matrix.
As a self-adhesive system it is possible to dispense with addition of further adhesives, which under certain circumstances give rise to side effects such as maceration, inflammation of the dermal areas, reduction of cutaneous respiration, etc.
Polyurethanes prove extremely advantageous over other adhesive materials, such as polyacrylates, rubber, etc., since they constitute no allergenic potential.
Polyurethane exhibits very good water vapor permeability. This ensures that, in the case of application for a prolonged period, there is no maceration through the release of water by the skin.
The oxygen permeability of polyurethane ensures a good supply of oxygen to the covered skin site, thereby countering damage to the tissue.
Polyurethane is allergenically neutral, so that following application there is no likelihood of allergic reactions by the body.
In contrast to other materials such as hydrocolloids or hydrogels, for example, polyurethane, moreover, shows no tendency to disintegrate on prolonged contact with fluids such as wound exudate. Consequently, on prolonged contact with wound fluid, a wound dressing produced from polyurethane does not leave residues in the wound that interfere with further wound healing.
Self-adhesive polyurethane disbonds on contact with fluid, so that sticking to newly formed tissue is prevented and, moreover, painless detachment of the wound cover is ensured.
Polyurethane wound contact materials of the invention produce a moist wound milieu, leading to more rapid wound healing.

The polymer matrix, preferably the polyurethane matrix, may be used with no foaming and/or with partial or full-area foaming, with no filling or with additional fillers, such as, for example, titanium dioxide, zinc oxide, plasticizers, dyes, etc.

Foaming of the matrix allows an improved cushioning effect to be achieved and, together with this an improved tactile sensation for the user.

The matrix, in particular the polyurethane polymer, may optionally comprise additives known per se from polyurethane chemistry, such as, for example, fillers and short, organic- or inorganic-based fibers, metal pigments, surface-active substances or liquid extenders such as substances having a boiling point of more than 150° C.

Examples of inorganic fillers that may be mentioned include heavy spar, chalk, gypsum, kieserite, sodium carbonate, titanium dioxide, cerium oxide, quartz sand, kaolin, carbon black and polar microspheres.

Organic fillers which can be used include, for example, powders based on polystyrene, polyvinyl chloride, urea-formaldehyde and polyhydrazodicarbonamide. Suitable short fibers include, for example, glass fibers 0.1-1 mm in length or fibers of organic origin, such as polyester fibers or polyamide fibers, for example. Metal powders, such as iron, aluminum, or copper powder, for example, may likewise be used in the context of gel formation. In order to give the gels the desired coloration it is possible to use the organic- or inorganic-based color pigments or dyes which are known per se in connection with the coloring of polyurethanes, such as, for example, iron oxide pigments or chromium oxide pigments, phthalocyanine-based or monoazo-based pigments. Surface-active substances include, for example, cellulose powders, activated carbon, and silica products.

To modify the adhesive properties of the gels it is possible where appropriate to make additions of polymeric vinyl compounds, polyacrylates, and other copolymers customary in adhesive technology, and/or adhesives based on natural substances, in an amount of up to 10% by weight, based on the weight of the gel composition, without altering the advantageous properties of the polyurethanes.

The polymer matrix can advantageously be made transparent. As transparent, water vapor permeable, and adhesive, the matrix thus fulfills esthetic and application-friendly aspects. This represents a significant advantageous difference from the polyacylate- and silicon gel-based plaster systems. Moreover, the transparency increases user acceptance, since the skin or wound contact materials of the invention, particularly in the form of patches or plasters, can be worn on the skin typically for a longer time period.

If the contact material of the invention is self-adhesive there is no need for additional fixing means. The wound contact material is placed directly as a dressing material on the wound to be covered, and by virtue of its self-adhesive properties adheres to the skin surrounding the wound.

In the case of sizeable wounds, if additional adhesive bonding is desired, or if the polymer matrix is not self-adhesive, the wound contact material can be adhered to the skin by the addition of an edge layer bonding system.

In that case the dressing material of the invention is constructed in accordance with known wound dressings. They are composed, generally speaking, of a backing material provided on one side with a self-adhesive layer. The wound contact material of the invention is then applied to this self-adhesive coating. In order to ensure ease of handling, a self-adhesive coating is additionally lined with a protective layer—a sealing paper, for example.

A suitable adhesive for the edge layer bonding system over the additional backing material is set out in DE 27 43 979 C3; in addition, the acrylate-based or rubber-based pressure-sensitive adhesives that are commercially customary can be used with preference for the adhesive coating.

Particular preference is given to thermoplastic hot-melt adhesives based on natural and synthetic rubbers and on other synthetic polymers such as acrylates, methacrylates, polyurethanes, polyolefins, polyvinyl derivatives, polyesters or silicones with appropriate adjuvants such as tackifier resins, plasticizers, stabilizers, and other auxiliaries where appropriate. If desired, post-crosslinking by UV or electron beam irradiation may be appropriate.

Hot-melt adhesives based on block copolymers, in particular, are distinguished by their multifarious possibilities for variation, since through the controlled reduction in the glass transition temperature of the self-adhesive composition, by virtue of the selection of the tackifiers, the plasticizers, and the polymer molecule size, and the molecular distribution of the components employed, the necessary functional bonding with the skin is ensured even at critical locations of the human locomotor apparatus.

The high shear strength of the hot-melt adhesive is achieved through the high cohesiveness of the polymer. The good tack results from the range of tackifiers and plasticizers that is employed.

The adhesive preferably includes at least one aromatic component with a fraction of less than 35%, preferably 5% to 30%.

For particularly strongly adhering systems the hot-melt adhesive is based preferably on block copolymers, especially A-B or A-B-A block copolymers or mixtures thereof. The hard phase A is principally polystyrene or its derivatives, and the soft phase B comprises ethylene, propylene, butylene, butadiene, isoprene or mixtures thereof, more preferably ethylene and butylene or mixtures thereof.

The controlled blending of di-block and tri-block copolymers is particularly advantageous, and in this case a di-block copolymer fraction of less than 80% by weight is preferred.

In one advantageous version the hot-melt adhesive has the composition indicated below:

| | |
|---|---|
| 10% to 90% by weight of | block copolymers |
| 5% to 80% by weight of | tackifiers such as oils, waxes, resins and/or |
| | mixtures thereof, preferably mixtures of |
| | resins and oils, |
| less than 60% by weight of | plasticizers, |
| less than 15% by weight of | additives, |
| less than 5% by weight of | stabilizers. |

The aliphatic or aromatic oils, waxes and resins serving as tackifiers are preferably hydrocarbon oils, waxes and resins, the consistency of the oils, such as paraffinic hydrocarbon oils, or of the waxes, such as paraffinic hydrocarbon waxes, having a favorable effect on bonding to the skin. Plasticizers used are medium- or long-chain fatty acids and/or their esters. These additions serve to set the adhesive properties and the stability. If desired, further stabilizers and other auxiliaries are employed.

The backing materials are composed preferably of an air and water vapor permeable but water impermeable polymer layer having a thickness of approximately 10 to 100 μm. The backing sheet, which is flexible in certain circumstances, is composed preferably of polymers of polyurethane, PE, PP, polyamide, polyester or polyether ester or of known backing materials such as wovens, nonwovens, foams, plastics, etc.

The polyurethane matrix of the invention may be applied atop this backing layer or backing sheet, in the way which is known from the prior art. In that case the matrix is lined on one side with the backing material and applied as a composite sheet. Depending on the backing material used it is possible by this means to control the water vapor permeability, the strength of the wound cover, the cushioning against pressure, and other physical qualities of the wound cover.

An inventively furnished dressing material, with or without additional edge bonding system, is then placed on the wound in customary fashion.

The direct introduction of numerous wound healing promoter substances and/or skincare substances into the matrix, in particular into the polymer matrix, is not possible prior to the crosslinking reaction thereof, since these substances possess active hydrogen atoms (hydroxyl, amino or acid groups) which would co-react in the crosslinking reaction, during polyurethane formation for example. The consequences would be an under-crosslinked matrix and covalently bonded actives no longer available for wound healing or skin care.

This problem can be remedied in accordance with the invention by introducing the wound healing promoter or skin care actives into the reaction mixture in encapsulated form at the same time removing them from the crosslinking reaction.

For this purpose the substances are bound in or encapsulated by means of water-absorbing polymers, such as superabsorbers, for example, this being referred to collectively also as incorporation.

Superabsorbers in which active or other substances such as dexpanthenol, for example, are in encapsulated form are, by way of example, crosslinked sodium polyacrylates of the kind known, for example, as Favor T®. They are composed of a crosslinked polyacrylate matrix in which, depending on its type, the active substance in question has been introduced into the matrix before or after the polymerization and can be released again from said matrix only in a swelling operation. These products can be incorporated into the non-crosslinked polyurethane matrix base materials prior to reaction, without inhibiting the crosslinking reaction of the polyurethane matrix. The active-doped superabsorber only then releases the active from the crosslinked matrix during application, i.e. on contact with aqueous media, such as the wound exudate, for example, over a relatively long time period and, with advantage, constantly.

With regard to the skin or wound contact materials of the invention it is preferred for the water-absorbing polymer to comprise

(α1) from 0.1% to 99.999%, preferably from 20% to 98.99%, and more preferably from 30% to 98.95% by weight of polymerized ethylenically unsaturated, acid-functional monomers or salts thereof, or polymerized, ethylenically unsaturated monomers containing a protonated or quaternized nitrogen, or mixtures thereof, particular preference being given to mixtures comprising at least ethylenically unsaturated, acid-functional monomers, preferably acrylic acid,

(α2) 0 to 70%, preferably from 1% to 60%, and more preferably from 1% to 40% by weight of polymerized, ethylenically unsaturated monomers copolymerizable with (α1),

(α3) from 0.001% to 10%, preferably from 0.01% to 7%, and more preferably from 0.05% to 5% by weight of one or more crosslinkers,

(α4) 0 to 30%, preferably from 1% to 20%, and more preferably from 5% to 10% by weight of water-soluble polymers, and

(α5) 0 to 20%, preferably from 0.01% to 7%, and more preferably from 0.05% to 5% by weight of one or more auxiliaries, the sum of the amounts by weight of (α1) to (α5) being 100% by weight.

The monoethylenically unsaturated, acid-functional monomers (α1) may be partly or fully neutralized, preferably partly neutralized. The degree of neutralization of the monoethylenically unsaturated, acid-functional monomers is preferably at least 25 mol %, more preferably at least 50 mol %, and with further preference 50-90 mol %. The monomers (α1) may be neutralized before and also after the polymerization. Furthermore, neutralization may take place with alkali metal hydroxides, alkaline earth metal hydroxides, ammonia, and carbonates and bicarbonates. In addition to these, any other base which forms a water-soluble salt with the acid is conceivable. Also conceivable is mixed neutralization with different bases. Preference is given to neutralization with ammonia or with alkali metal hydroxides, more preferably with sodium hydroxide or with ammonia.

Preferred monoethylenically unsaturated, acid-functional monomers (α1) are acrylic acid, methacrylic acid, ethacrylic acid, α-chloroacrylic acid, α-cyanoacrylic acid, β-methylacrylic acid (crotonic acid), α-phenylacrylic acid, β-acryloyloxypropionic acid, sorbic acid, α-chlorosorbic acid, 2′-methylisocrotonic acid, cinnamic acid, p-chlorocinnamic acid, p-stearylic acid, itaconic acid, citraconic acid, mesaconic acid, glutaconic acid, aconitic acid, maleic acid, fumaric acid, tricarboxyethylene, and maleic anhydride, acrylic acid and methacrylic acid being particularly preferred and acrylic acid being even more preferred.

Besides these carboxylate-functional monomers, preferred monoethylenically unsaturated, acid-functional monomers (α1) further include ethylenically unsaturated sulfonic acid monomers or ethylenically unsaturated phosphonic acid monomers.

Ethylenically unsaturated sulfonic acid monomers are allylsulfonic acid or aliphatic or aromatic vinylsulfonic acids or acrylic or methacrylic sulfonic acids. Preferred aliphatic or aromatic vinylsulfonic acids are vinylsulfonic acid, 4-vinylbenzylsulfonic acid, vinyltoluenesulfonic acid, and styrenesulfonic acid. Acrylic and methacrylicsulfonic acids are sulfoethyl (meth)acrylate, sulfopropyl (meth)acrylate, and 2-hydroxy-3-methacryloyloxypropylsulfonic acid. A preferred (meth)acrylamido alkylsulfonic acid is 2-acrylamido-2-methylpropanesulfonic acid.

Preference is further given to ethylenically unsaturated phosphonic acid monomers, such as vinylphosphonic acid, allylphosphonic acid, vinylbenzylphosphonic acid, (meth)acrylamido alkylphosphonic acids, acrylamido alkyldiphosphonic acids, phosphonomethylated vinylamines, and (meth)acrylophosphonic acid derivatives.

According to the present invention it is preferred for the water-absorbing polymer to be composed of at least 50% by weight, preferably at least 70% by weight, and more preferably at least 90% by weight of monomers containing carboxylate groups. It is particularly preferred in accordance with the invention for the water-absorbing polymer to be composed of at least 50% by weight, preferably at least 70% by weight, of acrylic acid, which is neutralized preferably to at least 20 mol %, more preferably to at least 50 mol %, and with further preference in the range from 65 to 85 mol %, preferably with sodium hydroxide solution.

As ethylenically unsaturated monomers (α1) containing a protonated nitrogen preference is given preferably to dialkylaminoalkyl (meth)acrylates in protonated form, examples being dimethylaminoethyl (meth)acrylate hydrochloride or dimethylaminoethyl (meth)acrylate hydrosulfate, and also to dialkylaminoalkyl(meth)acrylamides in protonated form, examples being dimethylaminoethyl(meth)acrylamide hydrochloride, dimethylaminopropyl-(meth)acrylamide hydrochloride, dimethylaminopropyl(meth)acrylamide hydrosulfate or dimethylaminoethyl)(meth)acrylamide hydrosulfate.

Preferred ethylenically unsaturated monomers (α1) containing a quaternized nitrogen are dialkylammonioalkyl (meth)acrylate in quaternized form, examples being trimethylammonioethyl (meth)acrylate methosulfate or dimethylethylammonioethyl (meth)acrylate ethosulfate, and also (meth)acrylamidoalkyldialkylamines in quaternized form, examples being (meth)acrylamidopropyltrimethylammonium chloride, trimethylammonioethyl (meth)acrylate chloride or (meth)acrylamido-propyltrimethylammonium sulfate.

Preferred monoethylenically unsaturated monomers (α2) which can be copolymerized with (α1) are acrylamides and methacrylamides.

Possible (meth)acrylamides, beside acrylamide and methacrylamide, include alkyl-substituted (meth)acrylamides or aminoalkyl-substituted derivatives of (meth)acrylamide, such as N-methylol(meth)acrylamide, N,N-dimethylamino-(meth)acrylamide, dimethyl(meth)acrylamide or diethyl(meth)acrylamide. Examples of possible vinyl amides are N-vinyl amides, N-vinylformamides, N-vinylacetamides, N-vinyl-N-methylacetamides, N-vinyl-N-methylformamides and vinylpyrrolidone. Particularly preferred among these monomers is acrylamide.

Further preferred monoethylenically unsaturated monomers (α2) which can be copolymerized with (α1) are water-dispersible monomers. Preferred water-dispersible monomers are acrylic esters and methacrylic esters, such as methyl (meth)acrylate, ethyl (meth)acrylate, propyl (meth)acrylate or butyl (meth)acrylate, and also vinyl acetate, styrene and isobutylene.

Crosslinkers (α3) which are preferred according to the present invention include compounds having at least two ethylenically unsaturated groups within one molecule (crosslinker class I), compounds having at least two functional groups which are able to react with functional groups of the monomers (α1) or (α2) in a condensation reaction (=condensation crosslinkers), in an addition reaction or in a ring-opening reaction (crosslinker class II), compounds which contain at least one ethylenically unsaturated group and at least one functional group which is able to react with functional groups of the monomers (α1) and (α2) in a condensation reaction, in an addition reaction or in a ring-opening reaction (crosslinker class II), or polyvalent metal cations (crosslinker class IV). The compounds of crosslinker class I produce crosslinking of the polymers through the free-radical polymerization of the ethylenically unsaturated groups of the crosslinker molecule with the monoethylenically unsaturated monomers (α1) or (α2), whereas in the case of the compounds of crosslinker class II and the polyvalent metal cations of crosslinker class IV the crosslinking of the polymers is achieved through condensation reaction of the functional groups (crosslinker class II) or by electrostatic interaction of the polyvalent metal cation (crosslinker class IV) with the functional groups of the monomers (α1) or (α2). In the case of the compounds of crosslinker class III, accordingly, crosslinking of the polymer takes place both by free-radical polymerization of the ethylenically unsaturated groups and by condensation reaction between functional groups of the crosslinker and the functional groups of the monomers (α1) or (α2).

Preferred compounds of crosslinker class I are poly(meth)acrylic esters which are obtained, for example, by the reaction of a polyol, such as ethylene glycol, propylene glycol, trimethylolpropane, 1,6-hexanediol, glycerol, pentaerythritol, polyethylene glycol or polypropylene glycol, for example, of an amino alcohol, of a polyalkylene polyamine, such as diethylentriamine or triethylenetetraamine, for example, or of an alkoxylated polyol with acrylic acid or methacrylic acid. Preferred compounds of crosslinker class I further include polyvinyl compounds, poly(meth)allyl compounds, (meth)acrylic esters of a monovinyl compound or (meth)acrylic esters of a mono(meth)allyl compound, preferably of the mono(meth)allyl compounds of a polyol or of an amino alcohol. Attention is drawn in this context to DE 195 43 366 and DE 195 43 368. The disclosures thereof are hereby incorporated by reference and are therefore considered part of the present disclosure.

Examples of compounds of crosslinker class I include alkenyl di(meth)acrylates, examples being ethylene glycol di(meth)acrylate, 1,3-propylene glycol di(meth)acrylate, 1,4-butylene glycol di(meth)acrylate, 1,3-butylene glycol di(meth)acrylate, 1,6-hexanediol di(meth)acrylate, 1,10-decanediol di(meth)acrylate, 1,12-dodecanediol di(meth)acrylate, 1,18-octadecanediol di(meth)acrylate, cyclopentanediol di(meth)acrylate, neopentyl glycol di(meth)acrylate, methylene di(meth)acrylate or pentaerythritol di(meth)acrylate, alkenyl di(meth)acrylamides, examples being N-methyl di(meth)acrylamide, N,N′-3-methylbutylidenebis(meth)acrylamide, N,N′-(1,2-dihydroxyethylene)bis(meth)acryl-amide, N,N′-hexamethylenebis(meth)acrylamide or N,N′-methylenebis(meth)-acrylamide, polyalkoxy di(meth)acrylates, examples being diethylene glycol di(meth)acrylate, triethylene glycol di(meth)acrylate, tetraethylene glycol di(meth)acrylate, dipropylene glycol di(meth)acrylate, tripropylene glycol di(meth)acrylate or tetrapropylene glycol di(meth)acrylate, bisphenol A di(meth)acrylate, ethoxylated bisphenol A di(meth)acrylate, benzylidine di(meth)acrylate, 1,3-di(meth)acryloyloxypropan-2-ol, hydroquinone di(meth)acrylate, di(meth)acrylate esters of trimethylolpropane alkoxylated, preferably ethoxylated, with 1 to 30 mol of alkylene oxide per hydroxyl group, thioethylene glycol di(meth)acrylate, thiopropylene glycol di(meth)acrylate, thiopolyethylene glycol di(meth)acrylate, tiopolypropylene glycol di(meth)acrylate, divinyl ethers, for example, 1,4-butanediol divinyl ether, divinyl esters, for example, divinyl adipate, alkane dienes, for example, butadiene or 1,6-hexadiene, divinylbenzene, di(meth)allyl compounds, for example, di(meth)allyl phthalate or di(meth)allyl succinate, homopolymers and copolymers of di(meth)allyldimethylammonium chloride and homopolymers and copolymers of diethyl(meth)allylaminomethyl(meth)acrylate ammonium chloride, vinyl-(meth)acrylic compounds, examples being vinyl (meth)acrylate, (meth)allyl(meth)acrylic compounds, examples being (meth)allyl (meth)acrylate, (meth)allyl (meth)acrylate ethoxylated with 1 to 30 mol of ethylene oxide per hydroxyl group, di(meth)allyl esters of polycarboxylic acids, examples being di(meth)allyl maleate, di(meth)allyl fumarate, di(meth)allyl succinate or di(meth)allyl terephthalate, compounds having 3 or more ethylenically unsaturated, free-radically polymerizable groups such as, for example, glycerol tri(meth)acrylate, (meth)acrylate esters of glycerol ethoxylated with preferably 1 to 30 mol of ethylene oxide per hydroxyl group, trimethylolpropane tri(meth)acrylate, tri(meth)acrylate esters of trimethylolpropane alkoxylated, preferably ethoxylated, with preferably 1 to 30 mol of alkylene oxide per hydroxyl group, trimethacrylamide, (meth)allylidene di(meth)acrylate, 3-allyloxy-1,2-propanediol di(meth)acrylate, tri(meth)allyl cyanurate, tri(meth)allyl isocyanurate, pentaerythritol tetra(meth)acrylate, pentaerythritol tri(meth)acrylate, (meth)acrylic esters of pentaerythritol ethoxylated preferably with 1 to 30 mol of ethylene oxide per hydroxyl group, tris(2-hydroxyethyl) isocyanurate tri(meth)acrylate, trivinyl trimellitate, tri(meth)allylamine, di(meth)allylalkylamines, for example, di(meth)allylmethylamine, tri(meth)allyl phosphate, tetra(meth)allyl-ethylenediamine, poly(meth)allyl esters, tetra(meth)allyloxyethane or tetra(meth)allyl-ammonium halides. Preferred according to the present invention in crosslinker class I are vinyl isocyanates, trivinyl trimellitate or tri(meth)allyl isocyanurate, with trivinyl trimellitate being particularly preferred.

Preferred compounds of crosslinker class II are compounds having at least two functional groups which are able to react in a condensation reaction (=condensation crosslinkers), in an addition reaction or in a ring-opening reaction with the functional groups of monomers (α1) or (α2), preferably with acid groups of the monomers (α1). These functional groups of the compounds of crosslinker class II are preferably alcohol, amine, aldehyde, glycidyl, isocyanate, carbonate or epichloro functions.

Examples of compounds of crosslinker class II include polyols, examples being ethylene glycol, polyethylene glycols, such as diethylene glycol, triethylene glycol, and tetraethylene glycol, propylene glycol, propylene glycols such as dipropylene glycol, tripropylene glycol or tetrapropylene glycol, 1,3-butanediol, 1,4-butanediol, 1,5-pentanediol, 2,4-pentanediol, 1,6-hexanediol, 2,5-hexanediol, glycerol, polyglycerol, trimethylolpropane, polyoxypropylene, oxyethylene-oxypropylene block copolymers, sorbitan fatty acid esters, polyoxyethylenesorbitan fatty acid esters, pentaerythritol, polyvinyl alcohol and sorbitol, amino alcohols, examples being ethanolamine, diethanolamine, triethanolamine or propanolamine, polyamine compounds, examples being ethylenediamine, diethylenetriamine, triethylenetetramine, tetraethylenepentamine or pentaethylenehexamine, polyglycidyl ether compounds such as ethylene glycol diglycidyl ether, polyethylene glycol diglycidyl ether, glycerol diglycidyl ether, glycerol polyglycidyl ether, pentaerythritol polyglycidyl ether, propylene glycol diglycidyl ether, polypropylene glycol diglycidyl ether, neopentyl glycol diglycidyl ether, hexanediol glycidyl ether, trimethololpropane polyglycidyl ether, sorbitol polyglycidyl ether, phthalic acid diglycidyl ester, adipic acid diglycidyl ether, 1,4-phenylenebis(2-oxazoline), glycidol, polyisocyanates, preferably diisocyanates such as toluene 2,4-diisocyanate and hexamethylene diisocyanate, polyazridine compounds such as 2,2-bishydroxymethylbutanol tris[3-(1-aziridinyl)propionate], 1,6-hexamethylenediethyleneurea, and diphenylmethane bis-4,4′-N-N′-diethyleneurea, halogen epoxides, examples being epichlorohydrin and epibromohydrin and α-methylepichlorohydrin, alkylene carbonates such as 1,3-dioxolan-2-one (ethylene carbonate), 4-methyl-1,3-dioxolan-2-one (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxan-2-one, 4-methyl-1,3-dioxan-2-one, 4,6-dimethyl-1,3-dioxan-2-one, 1,3-dioxolan-2-one, poly-1,3-dioxolan-2-one, and polyquaternary amines such as condensation products of dimethylamines and epichlorohydrin. Further preferred compounds of crosslinker class II are polyoxazolines such as 1,2-ethylenebisoxazoline, crosslinkers with silane groups, such as γ-glycidoxypropyltrimethoxysilane and γ-amino-propyltri-methoxysilane, oxazolidinones such as 2-oxazolidinone, bis- and poly-2-oxazolidinones and diglycol silicates.

Preferred compounds of class III are hydroxyl or amino containing esters of (meth)acrylic acid, such as 2-hydroxyethyl (meth)acrylate for example, and also hydroxyl or amino containing (meth)acrylamides, or mono(meth)allyl compounds of diols.

The polyvalent metal cations of crosslinker class IV are derived preferably from monovalent or polyvalent cations, the monovalent ones in particular from alkali metals, such as potassium, sodium or lithium, preference being given to lithium. Preferred divalent cations derive from zinc, beryllium, alkaline earth metals, such as magnesium, calcium or strontium, preference being given to magnesium. Higher polyvalent cations which can be used further in accordance with the invention are cations of aluminum, iron, chromium, manganese, titanium, zirconium, and other transition metals, and also double salts of such cations or mixtures of the stated salts. It is preferred to use aluminum salts and alums and their various hydrates such as, for example, AlCl₃×6H₂O, NaAl(SO₄)₂×12H₂O, KAl(SO₄)₂×12H₂O or Al₂(SO₄)₃×14-18H₂O.

With particular preference Al₂(SO₄)₃ and its hydrates are used as crosslinkers of crosslinking class IV.

Preference is given to water-absorbing polymers crosslinked by crosslinkers of the following crosslinker classes or by crosslinkers of the following combinations of crosslinker classes: I, II, III, IV, I II, I III, I IV, I II III, I II IV, I III IV, II III IV, II IV or III IV. The above combinations of crosslinker classes each constitute one preferred embodiment of crosslinkers of a water-absorbing polymer.

Corresponding to a further preferred embodiment are water-absorbing polymers crosslinked by any one of the abovementioned crosslinkers of crosslinker classes 1. Preferred among these are water-soluble crosslinkers. In this context, N,N′-methylenebisacrylamide, polyethylene glycol di(meth)acrylates, triallylmethylammonium chloride, tetraallylammonium chloride, and allyl nonaethylene glycol acrylate prepared with 9 mol of ethylene oxide per mole of acrylic acid, are particularly preferred.

As water-soluble polymers (α4) it is possible for water-soluble addition polymers, such as partly or fully saponified polyvinyl alcohol, polyvinylpyrrolidone, starch or starch derivatives, polyglycols or polyacrylic acid to be present, preferably in copolymerized form, in the absorbing polymers of the invention. The molecular weight of these polymers is not critical, provided they are water-soluble. Preferred water-soluble polymers are starch or starch derivatives or polyvinyl alcohol. The water-soluble polymers, preferably synthetic ones such as polyvinyl alcohol, may also serve as a graft base for the monomers to be polymerized.

Auxiliaries (α5) employed are preferably standardizers, odor binders, surface-active agents or antioxidants.

From the aforementioned monomers and crosslinkers it is possible to prepare the water-absorbing polymer by a variety of modes of polymerization, which are known to the skilled worker.

Typical processes are described in the following patents: U.S. Pat. No. 4,286,082, DE 27 06 135, U.S. Pat. No. 4,076,663, DE 35 03 458, DE 40 20 780, DE 42 44 548, DE 43 23 001, DE 43 33 056 and DE 44 18 818. The disclosures thereof are hereby incorporated reference and are therefore considered part of the present disclosure. In the case of the abovementioned types of polymerization it is preferred to introduce the wound healing substance actually together with the monomer or monomers or solvent or solvents employed in the corresponding polymerization processes, as a mixture, into the polymerization in the variants already described above.

By “wound healing substance” is meant in accordance with the invention, preferably, a substance or a mixture of substances, said substance, or at least one substance of said mixture, containing as a functional group a double bond, an OH group, an NH group or a COOH group, or a salt of at least one of these groups, preferably an OH group. It is preferred, moreover, for the wound healing substance to have from 2 to 100 carbon atoms and one to 20 oxygen atoms. The above properties are likewise preferred for the active substances or active drug substances of the invention.

Generally speaking, wound healing substances used that are based on plant extracts are Equisetum arvense, Aloe barbadensis, Arnica Montana, Arnica chamissonis, Symphytum officinale, Solanum dulcamara, Echinacea pallida, Potentilla erecta, Trigonella foenumgraecum, Juglans regia, Linum usitatissimum, Terminalia sericea, Oenothera biennis, Centella asiatica, Arctium lappa, Capsella bursa-pastoris, Hypericum perforatum, Matricaria recutita, Chamomille recutita, Agrimonia eupatoria, Centaurea cyanus, Larrea tridentate, Populus spec,. Echinacea pupurea, Calendula officinalis, Aesculus hippocastanum, Salvia officinalis, Plantago lanceolata, Quercus robur, Glycyrhiza glabra, Quercus petraea, Hamamelis virgian, Cardiospermum halicacabum, Betula, Urtica dioica, Buxus chinensis, Lavandula angustifolia, Lavandula hybrida, Crocus sativus, Smilax aspera, Melaleuca alternifolia, amino acids or Viola tricolor or the salts thereof or derivatives or mixtures of at least two thereof, in accordance with the invention.

Further suitable, additional wound healing substances or skin care agents are vitamins and the like, such as glucosamine sulfate, allantoin, biotin, chondroitin sulfate, coenzyme Q10, dexpanthenol, honey/honey extract, niacinamide, propolis, vitamin A or its esters, vitamin C and its esters, vitamin E and its esters or the salts thereof or derivatives or mixtures of at least two thereof, in accordance with the invention.

Wound healing substances are preferably, in accordance with the invention, dexpanthenol or extracts of marigold, preferably calendula oil; or witch hazel, preferably D-hamelose; or of camomile, preferably camomile blossom oil—preferably bisabolol or azulene—or mixtures of at least two of all of the above substances.

It is further preferred for in each case one of the above wound healing substances to be able to be present as the main component in a mixture, this main component being able to be present preferably at at least 50% by weight, more preferably at least 70% by weight, and very preferably at least 95% by weight, based in each case on the mixture.

Skin care substances chosen are preferably vitamins, antioxidants, photo protectants, insect repellants, essential oils, antimicrobial agents, moisturizers, perfumes, and, in particular, coenzyme Q10.

The wound healing promoter and/or skin care substances, which may be used individually or as a mixture, are included advantageously at from 0.1 to 10.0% by weight, preferably from 0.2% to 5% by weight, based on the polymer matrix, or at from 0.001 to 30% by weight, preferably from 5% to 15% by weight, based on the active-doped, water-absorbing polymer.

The incorporation of the wound healing or skin care substance into the water-absorbing polymer before the end of the formation, or before the beginning of the polymerization of the water-absorbing polymer may take place by means of the following process steps. On the one hand, the substance can be incorporated into the water-absorbing polymer via the solvent used for preparing the water-absorbing polymer. On the other, the substance can be added to the monomer, oligomer or prepolymer, or at least two thereof, that is or are used to form the water-absorbing polymer. In both of the above variants the wound healing or skin care substance may be in the form of a solution, emulsion or suspension. Furthermore, the two above variants can be combined with one another.

In another embodiment of the process of the invention the substance is incorporated after the end of the formation of the water-absorbing polymer or during its further processing, or both. This incorporation takes place preferably into a gel of the water-absorbing polymer. In that case it is preferred for the gel to have a water quantity, based on the water-absorbing polymer, of from 0.2 to 20 times, preferably from 1 to 10 times, and more preferably from 2 to 4 times, in order to achieve very highly uniform incorporation of the wound healing substance.

This can be achieved on the one hand by absorption of the substance by means of a liquid, generally aqueous, vehicle, in which the substance is preferably in solution. In the case of incorporation of the wound healing or skin care substance in the course of further processing it is preferred for the substance to be incorporated in a liquid, preferably aqueous, phase into the water-absorbing polymer, where appropriate during the “post-crosslinking”.

Combinations of the above process variants are also possible. In the case of incorporation of the substance before the end of the formation of the water-absorbing polymer, a uniform doping of the water-absorbing polymer is preferably achieved; in other words, advantageously, the substance is present homogeneously distributed in the polymer. If the substance is incorporated after the end of the formation of the water-absorbing polymer or during its further processing, or both, then preferably the doping of the water-absorbing polymer particle in its outer or surface region is like that illustrated in FIG. 2. Combining the two process variants leads, as a general rule, to a polymer having a different concentration in the inner and outer regions of the water-absorbing polymer particle, the concentration of wound healing or skin care substance generally being higher in the outer region.

The water-absorbing polymer thus obtained, doped with the substance, and referred to from now on as “doped or incorporated polymer”, can be incorporated subsequently into the polymer matrix, preferably into the polyurethane matrix. It is preferred for the doped polymer to be incorporated into the polycondensate matrix before the end of its formation, in other words before substantially all of the reactive functional groups of a polyurethane matrix monomer have been consumed by reaction. This is accomplished preferably by being able to add the doped polymer to the polyol needed for the formation of the polyurethane matrix.

The wound dressing of the invention allows the skilled worker, therefore, for the first time to supply the active substances right at the beginning of the polyurethane formation reaction. This offers him or her great advantages in respect of breadth of variation and amount of active substances employed, and increases the flexibility associated with the production of the wound dressings.

A wound dressing having the desired properties is obtained, for example, by coating out flatly a mixture of the following ingredients, crosslinked by means of an appropriate catalyst:

| | |
|---|---|
| Polyether polyol | 334 g |
| Crosslinker | 29 g |
| Superabsorber doped with 10% | 37 g |
| wound healing substance (e.g., dexpanthenol) | |
| Catalyst | 0.8 g |

If the wound dressing thus obtainable is applied to a wound, the wound fluid causes the water-absorbing polymer to swell. The wound fluid is taken up by the polyurethane matrix and the water-absorbing polymers present therein. In contact with the fluid, said polymers begin to swell. As a result of this swelling process, the wound healing promoter substance or substances is or are released into the water-absorbing polymers. This allows direct release of the active substance at the wound treatment site.

This is an exceptionally good application advantage, which allows an “intelligent bandage” to be produced, that releases the requisite active wound healing promoter substance only on contact with wound fluid.

The kinetics of the release can be controlled on the one hand by the concentration of wound healing promoter substance in the water-absorbing polymer and on the other hand via the concentration of water-absorbing polymer in the polyurethane matrix. The release is further influenced by the distribution of the substance in the water-absorbing polymer (FIG. 2). The distribution of the active substance in the water-absorbing polymer takes place in accordance with the invention, as described above, as a function of the time of addition before, after or during the end of the formation of the water-absorbing polymer. Preference is given to a wound dressing where the active substance is distributed, preferably homogeneously, throughout the water-absorbing polymer (FIG. 2, Version I).

Consequently, solely by way of dry storage, a wound dressing ready for use is available for the user, which as well as the known advantages of hydroactive polyurethane wound dressings develops its wound healing promoter effect only where the latter is needed.

Furthermore, the production method, the incorporation of the active substances via encapsulation in the superabsorber, is what makes it actually possible for the direct incorporation of wound healing promoter substances into the polyurethane matrix prior to its formation reaction. Only as a result of this is it at all possible to realize product structures which are doped homogeneously with active substances and whose shaping takes place during the crosslinking reaction.

It is further preferred for the water-absorbing polymer of the wound dressing to have at least one of the following properties:
A1) a particle size distribution with at least 80% by weight of the particles possessing a size in a range from 10 μm to 900 μm, as determined by ERT420.1-99;
A2) a centrifuge retention capacity (CRC) of at least 10 g/g, preferably at least 20 g/g, determined by ERT441.1-99;
A3) an absorption against pressure (AAP) at 0.7 psi of at least 4 g/g, determined by ERT442.1-99;
A4) a water-soluble polymer content after a 16 hour extraction of less than 25% by weight, based in each case on the total weight of the water-absorbing polymer, determined by ERT470.1-99;
A5) a residual moisture content of not more than 15% by weight, based in each case on the total weight of the water-absorbing polymer, determined by ERT430.1-99,
   the stated measurement methods for particle size determination being known.

Mention may be made, by way of example, of the following parameters for characterizing the wound contact material when 10% superabsorber is added:

| | | |
|---|---|---|
| adhesiveness: | rheological characterization | 0.20 |
| | of the viscoelastic | |
| | properties tanδ | |
| | (ω = 0.3 rad/s): | |
| fluid management: | fluid uptake: | 10 g/100 cm² |
| | water vapor permeability: | 250 g/(m² × 24 h) |
| | O₂ permeability: | 2000 cm³/(m² × 24 h) |

The above-mentioned properties can be determined using the test methods below.

ERT

Unless described otherwise below, ERT methods are used for determining the various properties pertaining to the water-absorbing polymer. ERT stands for EDANA recommended test, with EDANA standing for European Nonwoven and Diaper Association.

Extraction Test

0.5 g of an active-doped sample—for example, a partially neutralized polyacrylic acid with a low degree of crosslinking, containing dexpanthenol as active, or a polyurethane matrix comprising it, is weighed out into a 125 ml wide-neck bottle on an analytical balance. Following the addition of 100 ml of a 0.9% strength solution of common salt (based on distilled water) and one drop of concentrated phosphoric acid, the mixture is stirred at 350 revolutions per minute on a magnetic stirrer for an hour. Then 2 ml of the solution are withdrawn and filtered through a 0.45 μm mixed cellulose ester membrane filter into a sample vial. The filtrate is then passed to an HPLC analysis, the sample used in the HPLC analysis having an acid pH in the region of 2.5 to 3.0.

The amount of active is ascertained from the results of the HPLC analysis, using external calibration. For that purpose the active under determination is weighed in an amount of at least 10 mg to an accuracy of 0.1 mg, using an analytical balance, into a measuring flask with a capacity of 100 ml. The measuring flask is then filled to the mark with ultrapure water. Corresponding to the concentration of the resultant stock solution, a dilution series is then produced on the analytical balance. This dilution series is used to compile a calibration plot by means of HPLC analyses. The amount of active extracted over an hour is determined by comparing the HPLC analysis results for the active in question with the calibration plot.

The chromatographic conditions are optimized as a function of the active under determination. In the case of dexpanthenol the column used may be a GromSil 300 ODS-5 5 μm (250×4 mm) column. The eluent is prepared by weighing out 13.61 g of KH₂PO₄ into a glass beaker with a capacity of 3 l and carrying out dissolution following the addition of 2000 ml of ultrapure water. Concentrated phosphoric acid is then used to set a pH of 2.5 to 3.0. In the case of dexpanthenol the flow rate set is 0.8 ml/min. Injection takes place via a 20 μl loop.

Rheological Characterization

A sample with a diameter of 8 mm is punched out centrally from a bandage and is preconditioned at 23±2° C. and 50±5% rh for an hour. The sample is adhered centrally to an 8 mm rotating plate and measured on a shearing stress-controlled rheometer with a Peltier element for thermal conditioning (e.g., RS-75 from Haake). For that purpose the sample is pressed onto the bottom plate with a standard force of 1.3 N. After conditioning for 5 minutes at 25±0.2° C., the viscoelastic properties (storage modulus and loss modulus) are determined in the frequency range from φ=0.3 to 30 rad/s with a shearing stress of 700 Pa. The tanδ is calculated from the ratio of loss modulus to storage modulus.

Fluid Absorption

A sample with a diameter of 15 mm is punched out centrally from a bandage and preconditioned at 23±2° C. and 50±5% rh for an hour. The samples are weighed and immersed fully for 3 hours in physiological sodium chloride solution which has a temperature of 23±0.5° C. The samples are weighed again and the fluid absorption is calculated from the weight difference.

Water Vapor Permeability

The test is carried out in accordance with ASTM E 96 (water method), with the following differences:

The opening of the test vessel is 804 mm².

The material is preconditioned for 24 hours at 23±2° C. and 50±5% rh.

The distance between the water level in the test vessel and the sample is 35±5 mm.

The reweighing of the test vessels complete with samples is carried out after 24 hours, during which they are stored in a controlled-climate cabinet at 37±1.5° C. and 30±3% rh.

O2 Permeability

Test in accordance with ASTM D3985-8.

It is further preferred for the water-absorbing polymer to have a particle size distribution of between 10 and 500 μm and/or a residual moisture content of less than 10% by weight, preferably less than or equal to 3% by weight.

The above particle size distributions and particle sizes, and also residual moisture contents, are particularly advantageous for uniform delivery and distribution of the active substances and for good wear comfort. It has emerged, moreover, that the above particle size distributions and particle sizes can be incorporated particularly effectively into flexible matrices, which, when incorporated into plasters or wound contact materials, raises the conformability of these plasters or wound contact materials to the shape of the wound and to its movements.

Finally, the matrix may be lined with an adhesive-repellant backing material, such as siliconized paper, or may be provided with a wound contact material or cushioning. On its preferably self-adhesive side which later faces the skin, the dressing of the invention is lined over its whole width, up until the time of use, typically with an adhesive-repellant backing material. This material protects the self-adhesive layer, which comprises the highly skin-compatible adhesive of the matrix and has been applied preferably by the transfer method, and, additionally, stabilizes the entire product. The liner may be designed in a known way as a single piece or, preferably, in two parts.

The wound dressing of the invention, mostly in the form of a plaster, preferably comprises a self-adhesive polyurethane matrix of the invention, comprising active substance, an active-impermeable backing layer, and a detachable protective layer, which is removed prior to application to the skin. Further ingredients, such as filler, stabilizers, enhancers and/or cosmetic adjuvants, may be incorporated in the matrix in order to tailor the dressing to the different fields of application and in order to provide a dressing which is amiable in application.

For the purpose of explanation,

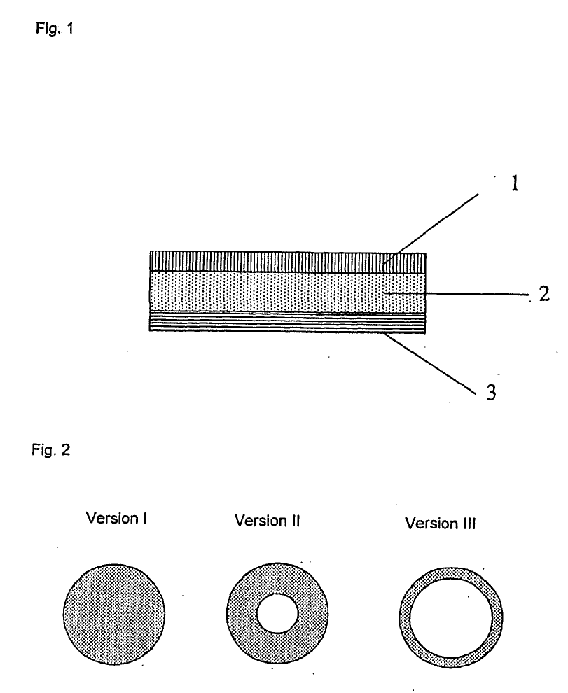

FIG. 1 shows one preferred embodiment of the wound dressing

FIG. 2 shows the distribution of the actives in the water-absorbing polymer in versions I, II and III (see examples).

A corresponding bandage is constructed from a backing such as films, nonwovens, wovens, foams (1) etc., the adhesive matrix (2), and liner sheet, liner material or release paper (3) to protect the adhesive matrix prior to use of the bandage, as depicted in FIG. 1.

In a further preferred embodiment of the invention, polymer sheets, nonwovens, wovens and combinations thereof are used as backings. Backing materials available for selection include polymers such as polyethylene, polypropylene, polyesters, polyether-esters and polyurethane, or else natural fibers. The thickness of the respective layers (1, 2, 3) is in the region of

(1) 10-150 μm

(2) 50-2000 μm

(3) 20-200 μm.

In summary it can be stated that suitable backing materials include all rigid and elastic sheet-like structures of synthetic and natural raw materials. Preference is given to backing materials which can be employed in such a way that they fulfill properties of a functional dressing. Recited by way of example are textiles such as wovens, knits, lays, nonwovens, laminates, nets, films, foams, and papers. Furthermore, these materials may be pretreated and/or aftertreated. Common pretreatments are corona and hydrophobizing; customary post-treatments are calendering, thermal conditioning, laminating, die-cutting, and enveloping.

It is particularly advantageous if the backing material is sterilizable, preferably γ (gamma) sterilizable. γ sterilization (dose=30 kGy) did not show any effect on the wound dressing of the invention.

The aforementioned properties of the adhesive matrix suggest in particular the use of the wound dressings of the invention for medical products, especially bandages, medical attachments, wound covers, and orthopedic or phlebological bandages, and dressings.

The wound dressings of the invention are capable of drawing up wound exudate and moisture from the skin and, where appropriate, of transporting it outward through the bandage. This produces an optimum moist wound healing environment. On the basis of the skin-compatibility and the painless redetachability, furthermore, preconditions important to the user for the use of the wound dressing of the invention are provided.

Besides its application as a dressing, plaster or bandage material, the contact material of the invention may also be employed as a skin care product. For that purpose it may incorporate not only the active-doped superabsorbents present in accordance with the invention but also further substances, especially skincare, skin-moisturizing or skin-healing substances. A further possible use of the contact material is as a moist or dry cosmetic wipe or pad.

### DETAILED DESCRIPTION OF THE INVENTION

### Examples

A. Production of Laboratory Specimens with Dexpanthenol-Doped Superabsorber

1. Preparation of the two Compositions

| | | |
|---|---|---|
| Component 1: | 82% by weight | polyether polyol* |
| | | Levagel E (Bayer AG) |
| | 9% by weight | isocyanate prepolymer** |
| | | Desmodur (Bayer AG) |
| | 9% by weight | Favor T, doped with 10% dexpanthenol |
| | | (Degussa Stockhausen AG) |

are weighed and mixed on a roller bed for 24 hours.

| | | |
|---|---|---|
| Component 2: | 90% by weight | polyether polyol* |
| | 10% by weight | catalyst*** |
| | | CosCat (CasChem Inc.) |

*Pentaerythritol + propylene oxide + ethylene oxide copolymer with ethylene oxide end block
Functionality: 4, OH number: 35, average molar weight: 6400 (calculated). Viscosity
(23° C.): 1000 mPas, ethylene oxide content: 20% by weight
**NCO-terminated prepolymer from reaction at 80° C. of hexamethylene diisocyanate and polypropylene glycol (average molecular weight: 220) in a molar ratio of 5:1 and subsequent vacuum distillation at approximately 0.5 mbar down to a residual HDI monomer content <0.5% by weight
NCO content: 12.6% by weight, viscosity (23° C.): 5000 mPas
***Solution of 1 mol of the Bi(III) salt with 2,2-dimethyloctanoic acid in 3 mol of 2,2-dimethyoctanoic acid (bismuth content approximately 17% by weight)

2. Production of Coatings

| Materials for use: | component 1 | |
|---|---|---|
| | component 2 | |
| | Sheet with a water vapor | |
| | permeability ofapproximately | |
| | 750 g/(m² * d) | |
| Procedure: | 98% by weight | component 1 |
| | 2% by weight | component 2 |

are weighed out and mixed for approximately 40 s, poured onto release paper, the film is laminated on ahead of the coating bar, and the PU composition is coated out between release paper and sheet, using the coating bar:
Slot setting on the coating bar: 1 mm

Curing of the coated-out PU composition at 65° C. for 5 minutes

From the coating (coat weight of approximately 850 g/m²) it is possible to die-cut contact materials.

Illustrated below by way of example is the production of a wound dressing of the invention.

A. Preparation of polyacrylic acid-based superabsorber doped with dexpanthenol The superabsorber is prepared by customary methods, by initiating the polymerization of aqueous acrylic acid solution at a temperature of approximately 150° C. The water content of the solution is approximately 70% by weight. Even at this point (I) it is already possible to add the dexpanthenol to the polymerization solution. The result is a dexpanthenol-doped superabsorber of version (I) as depicted in FIG. 2. The resultant polymer is comminuted and dried at approximately 150° C. It is likewise possible to add dexpanthenol (II) subsequently, hence giving a dexpanthenol-doped superabsorber of version (II). This later addition of dexpanthenol has the advantage that the dexpanthenol has not been exposed to the prior drying. The polymer is ground further and, where appropriate, surface-modified and dried. It is likewise possible to add the dexpanthenol at this point (III). This produces a dexpanthenol-doped superabsorber of version (III). The polymer is subsequently dried at approximately 150° C. down to a residual moisture content of approximately 7% to 10%.

Advantageously, two further process steps (IV and V) then follow, which optimize first the particle size and secondly the residual moisture content of the dexpanthenol-doped superabsorber.

Thereafter the dexpanthenol-doped polymer dried after process step (III) is ground. The superabsorber (IV) then has a particle size distribution of approximately 10 to 500 μm, preferably 20 to 200 μm.

As a last process step (V) the doped superabsorber is dried again. The drying leads to a residual moisture content of less then 10%, preferably ≦3%, in the doped superabsorber (V).

In comparison between the doped superabsorbers of versions I to V that can be prepared, a number of differences become apparent, as shown in FIG. 2. With the same amount of dexpanthenol employed, in the case of version (I) the dexpanthenol is homogeneously distributed within the superabsorber particles. In the case of version (II), the dexpanthenol is distributed in an outer ring of the particles, and in the case of version (III) all of the dexpanthenol is located only on the outermost layer of the particle surface. The result of the two latter versions (II) and (III) is that, owing to the accumulation of dexpanthenol at the surface, the superabsorber in part becomes more tacky and hence processing may become more difficult.

Version (IV), with a max. particle size of 500 μm and a minimum particle size above the pulmonary access level, represents the optimum particle size distribution. Hence effective further processing of the doped superabsorber particles is ensured.

Version (V), with a residual moisture content of less than 10%, is another optimized version of the dexpanthenol-doped superabsorber.

A superabsorber which has proven particularly preferable for use in the wound dressings of the invention, accordingly, is a dexpanthenol-doped superabsorber of the combination of versions I, IV and V. In this optimum doped superabsorber the dexpanthenol is homogeneously distributed within the particles of absorbent. The particles of the absorbent have a size distribution of between 10 and 500 μm and possess a residual moisture content of less than 10%, preferably less than or equal to 3%.

As a result of the different preparation options (version I, II and/or III in accordance with FIG. 2) a breadth of variation is created in the active substance release kinetics as well. Version I generates a long-lasting release, with, advantageously, homogeneous distribution of the active substance in the polymer. Through a combination of the individual preparation steps it is therefore possible to tailor release ranges of the active substances. Hence the active substance can be released in a relatively short time and high dose through to long-lasting delivery in a low dose.

B. Production of the Wound Dressing

These doped superabsorber particles are then added directly to the initial mixture for the polyurethane reaction. Not only the uniform distribution of the dexpanthenol in the superabsorber (version I) but also a residual moisture content of less than or equal to 3% (version V) do not cause any adverse effect, disruptive to the production operation, on the formation of the polyurethane. Polyurethane formation hence proceeds without disruption, as described above. The polyurethane matrix comprising the doped superabsorber is subsequently poured out onto release paper and lined with a polyurethane sheet.

This polyurethane matrix, enveloped between polyurethane sheet and release paper, is manufactured as bales and brought to the appropriate web width on master rolls. From the webs it is possible to die-cut the appropriate-sized wound contact materials. These self-adhesive wound contact materials can themselves be used as plasters and have, accordingly, a structure corresponding to FIG. 1.

In a further process step, these polyurethane wound contact materials with doped superabsorbers can be placed onto a backing material with an adhesive. From this backing material, finally, the finished bandage with adhesive margin of appropriate size can be die-cut. Suitable backing materials are the materials which are known in bandage technology, such as sheets of polyurethane, polyethylene, polypropylene, polyamide, polyester or polyether-ester and also wovens, fleece, nonwovens, knits, lays, laminates, nets, sheets, foams or papers. It is likewise possible to use any known adhesives, such as acrylate or hot-melt polyurethane.

The polyurethane sheet used in the production operation is optional. It is likewise possible to apply and further process the polyurethane matrix only on the release paper, which means that the polyurethane sheet is then absent from the finished wound dressing.

What is claimed is:

1. A skin or wound contact material comprising a polycondensate matrix and a water-absorbing polymer incorporated therein, the water-absorbing polymer being doped with at least one of a wound healing promoter substance and a skin care substance.

2. The skin or wound contact material of claim 1, wherein at least a part of the water-absorbing polymer is covalently bonded to the polycondensate matrix.

3. The skin or wound contact material of claim 1, wherein the polycondensate matrix is air and water vapor permeable.

4. The skin or wound contact material of claim 1, wherein the polycondensate matrix is self-adhesive.

5. The skin or wound contact material of claim 1, wherein the polycondensate matrix is transparent.

6. The skin or wound contact material of claim 1, wherein the polycondensate matrix comprises a polyurethane matrix.

7. The skin or wound contact material of claim 6, wherein the polyurethane matrix is formed from
(a) one or more polyether polyols having from 2 to 6 hydroxyl groups, OH numbers of from 20 to 112, and an ethylene oxide content of at least 10% by weight,
(b) one or more antioxidants,
(c) a catalyst comprising one or more bismuth (III) carboxylates which are based on carboxylic acids having from 2 to 18 carbon atoms and are soluble in the polyols (a), and
(d) hexamethylene diisocyanate.

8. The skin or wound contact material of claim 7, wherein a product of functionalities of components (a) and (d) is at least 5.2 and a ratio of free NCO groups of component (d) to free OH-groups of component (a) is from 0.30 to 0.70.

9. The skin or wound contact material of claim 7, wherein component (c) is present in an amount of from 0.005% to 0.25% by weight and component (b) is present in an amount of from 0.1% to 1.0% by weight, each based on component (a).

10. The skin or wound contact material of claim 1, wherein the water-absorbing polymer is present in particulate form.

11. The skin or wound contact material of claim 1, wherein the water-absorbing polymer comprises at least 50% by weight of one or more carboxylate group containing monomers.

12. The skin or wound contact material of claim 2, wherein the water-absorbing polymer comprises at least 70% by weight of one or more carboxylate group containing monomers.

13. The skin or wound contact material of claim 10, wherein the water-absorbing polymer comprises at least 90% by weight of one or more carboxylate group containing monomers.

14. The skin or wound contact material of claim 1, wherein the water-absorbing polymer comprises at least 50% by weight of acrylic acid and at least 20 mol % of the acrylic acid are neutralized.

15. The skin or wound contact material of claim 14, wherein at least 50 mol % of the acrylic acid are neutralized.

16. The skin or wound contact material of claim 15, wherein from 65 to 85 mol % of the acrylic acid are neutralized.

17. The skin or wound contact material of claim 1, wherein the water-absorbing polymer comprises crosslinked sodium polyacrylate.

18. The skin or wound contact material of claim 1, wherein the water-absorbing polymer exhibits one or more of
A1) a particle size distribution wherein at least 80% by weight of particles have a size of from 10 μm to 900 μm, determined according to ERT 420.1-99;
A2) a centrifuge retention capacity (CRC) of at least 10 g/g, determined according to ERT 441.1-99;
A3) an absorption against pressure (MP) at 0.7 psi of at least 4 g/g, determined according to ERT 442-1-99;
A4) a water-soluble polymer content after a 16 hour extraction of less than 25% by weight, based on a total weight of the water-absorbing polymer, determined according ERT 470.1-99;
A5) a residual moisture content of not more than 15% by weight, based on a total weight of the water-absorbing polymer, determined according ERT 430.1-99.

19. The skin or wound contact material of claim 18, wherein the water-absorbing polymer exhibits a CRC of at least 20 g/g.

20. The skin or wound contact material of claim 10, wherein the water-absorbing polymer exhibits at least one of a particle size distribution of from 10 μm to 500 μm and a residual moisture content of less than 10% by weight.

21. The skin or wound contact material of claim 1, wherein the water-absorbing polymer exhibits a residual moisture content of less than 3% by weight.

22. The skin or wound contact material of claim 1, wherein the at least one of a wound healing promoter substance and a skin care substance is present in an amount of from 0.001% to 30% by weight, based on a total weight of the water-absorbing polymer and the at least one of a wound healing promoter substance and a skin care substance.

23. The skin or wound contact material of claim 11, wherein the at least one of a wound healing promoter substance and a skin care substance is present in an amount of from 5% to 15% by weight, based on a total weight of the water-absorbing polymer and the at least one of a wound healing promoter substance and a skin care substance.

24. The skin or wound contact material of claim 1, wherein the at least one of a wound healing promoter substance and a skin care substance is present in an amount of from 0.1% to 10.0% by weight, based on a weight of the matrix.

25. The skin or wound contact material of claim 6, wherein the at least one of a wound healing promoter substance and a skin care substance is present in an amount of from 0.2% to 5% by weight, based on a weight of the matrix.

26. The skin or wound contact material of claim 1, wherein the at least one of a wound healing promoter substance and a skin care substance is distributed over the entire water-absorbing polymer.

27. The skin or wound contact material of claim 23, wherein the at least one of a wound healing promoter substance and a skin care substance is homogeneously distributed over the entire water-absorbing polymer.

28. The skin or wound contact material of claim 1, wherein the water-absorbing polymer is present in an amount of from 70% to 99.99% by weight, based on a total weight of the water-absorbing polymer and the at least one of a wound healing promoter substance and a skin care substance, the water-absorbing polymer comprises at least 90% by weight of a crosslinked polyacrylic acid, based on the water-absorbing polymer, and the crosslinked polyacrylic acid comprises at least 90% by weight, based on the crosslinked polyacrylic acid, of acrylic acid which comprises at least 30 mol % of partially neutralized acrylic acid.

29. The skin or wound contact material of claim 1, wherein the at least one of a wound healing promoter substance and a skin care substance exhibits an availability of at least 10% by weight.

30. The skin or wound contact material of claim 1, wherein the at least one of a wound healing promoter substance and a skin care substance comprises at least one of dexpanthenol, marigold, witch hazel, camomile, a vitamin, an antioxidant, a light stabilizer, an insect repellent, an essential oil, an antimicrobial agent, a moisturizer, a perfume and coenzyme Q10.

31. The skin or wound contact material of claim 11, wherein the at least one of a wound healing promoter substance and a skin care substance comprises at least one of dexpanthenol and coenzyme Q10.

32. A skin or wound contact material comprising a self-adhesive, air and water vapor permeable polyurethane matrix and a water-absorbing polymer incorporated therein, the water-absorbing polymer comprising at least 50% by weight of one or more carboxylate group containing monomers and being doped with at least one of a wound healing promoter substance and a skin care substance.

33. The skin or wound contact material of claim 32, wherein at least a part of the water-absorbing polymer is covalently bonded to the polyurethane matrix.

34. The skin or wound contact material of claim 32, wherein the water-absorbing polymer is present in particulate form.

35. The skin or wound contact material of claim 34, wherein the water-absorbing polymer comprises at least 50% by weight of acrylic acid and from 65 to 85 mol % of the acrylic acid are neutralized.

36. The skin or wound contact material of claim 35, wherein the water-absorbing polymer exhibits at least one of a particle size distribution of from 10 μm to 500 μm and a residual moisture content of less than 3% by weight.

37. The skin or wound contact material of claim 32, wherein the at least one of a wound healing promoter substance and a skin care substance comprises at least one of dexpanthenol, marigold, witch hazel, camomile, a vitamin, an antioxidant, a light stabilizer, an insect repellent, an essential oil, an antimicrobial agent, a moisturizer and coenzyme Q10.

38. The skin or wound contact material of claim 1, wherein the material further comprises a backing sheet.

39. The skin or wound contact material of claim 38, wherein the backing sheet comprises at least one of a polyurethane, a polyethylene, a polypropylene, a polyamide, a polyester and a polyether-ester.

40. The skin or wound contact material of claim 1, wherein the material further comprises at least one of a liner sheet, a liner paper and a release paper.

41. The skin or wound contact material of claim 1, wherein the material is comprised in a wound dressing, a bandage or a plaster.

42. A cosmetic wipe or pad which comprises the skin or wound contact material of claim 1.

43. A process for producing a skin or wound contact material which comprises a polyurethane matrix and a water-absorbing polymer which has at least one of a wound healing promoter substance and a skin care substance incorporated therein, the process comprising reacting a mixture comprising a polyether polyol and an aliphatic isocyanate prepolymer and adding a water-absorbing polymer doped with the at least one of a wound healing promoter substance and skin care substance to form the polyurethane matrix having the water-absorbing polymer incorporated therein.

44. The process of claim 43, wherein the process further comprises coating the polyurethane matrix having the water-absorbing polymer incorporated therein two-dimensionally onto a backing sheet.
